(19) United States
(12) Patent Application Publication
(10) Pub. No.: US 2007/0293827 A1
(43) Pub. Date: Dec. 20, 2007

(54) Observation system of giving an injection of ringer's solution

(76) Inventor: Mi-Kyung Kim, Pusan (KR)

Correspondence Address: DAFFER MCDANIEL LLP, P.O. BOX 684908, AUSTIN, TX, 78768, US

(21) Appl. No.: 11/301,335

(22) Filed: Dec. 12, 2005

Related U.S. Application Data
(63) Continuation of application No. PCT/KR04/01402, filed on Jun. 11, 2004.

(30) Foreign Application Priority Data
Jun. 13, 2003 (KR) 10-2003-0038184

(51) Int. Cl. A61M 5/00

(52) U.S. Cl. 604/246

(57) ABSTRACT

Disclosed herein is a system for monitoring the injection of Ringer's solution. The monitoring system of the present invention includes a status detection device for measuring the weight of Ringer's solution, converting the weight information of the Ringer's solution into a data signal, and transmitting the data signal. A first line interface device receives the data signal from the status detection device, performs data conversion with respect to the data signal to allow identifier information required to identify the status detection device to be included in the data signal, and outputs the converted data to the communication line. A second line interface device receives the data signal through the communication line, and outputs the data signal to a remote monitoring device. The remote monitoring device decodes the data signal and outputs weight data of the Ringer's solution according to the identifier information.

### CROSS REFERENCE TO RELATED APPLICATIONS

This application is a continuation of pending International Application No. PCT/KR2004/001402 filed Jun. 11, 2004, which designates the United States and claims priority to Korean Application No. 10-2003-0038184 filed on Jun. 13, 2003.

### BACKGROUND OF THE INVENTION

1. Field of the Invention

The present invention relates, in general, to a system for monitoring the injection of Ringer's solution, which can monitor the injection status of Ringer's solution to a patent at a remote place and, more particularly, to a system for monitoring the injection of Ringer's solution, which measures and outputs variations in weight of the Ringer's solution, transmits the output signal to a remote place, such as a nurse room, through a communication line, and displays the output signal, thus easily monitoring the injection status of the Ringer's solution at the remote place, such as the nurse room.

2. Description of the Related Art

Generally, when an injection of a nutritive solution is given to a patient or a lot of medicines are required to be given to a patent in hospital, intravenous injection using a Ringer bottle or pack is performed. Such an intravenous injection process requires a long time, and may cause shock, etc. if the amount of solution injected per unit time is large. Further, if air is injected into a solution tube after a solution has been completely injected, fatal danger to a patent may exist, so that there is the inconvenience of forcing a nurse or caregiver to periodically observe the status of the injection of Ringer's solution and the condition of a patient during the injection of the solution.

In order to solve the inconvenience, there was filed, in the prior art, a plurality of designs and patents for monitoring the injection of Ringer's solution using a plurality of Ringer bottles or packs, and outputting an alarm signal when the injection of the solution is completed. As examples of the prior art, there are a “solution observation apparatus” disclosed in Korean Utility Model Registration No. 195385 and designed to measure the number of drops of a solution using an infrared sensor and calculate the amount of remaining solution, an “electronic alarm and light emission signal mechanism” disclosed in Korean Utility Model Registration No. 302890 and designed to insert a solution level detection device into a container of Ringer's solution, detect the level of the solution, and output an alarm signal when the solution level reaches a certain level, and an “alarm device for detecting low level of Ringer's solution” disclosed in Korean Utility Model Registration No. 310677 and designed to measure the weight of Ringer's solution through a weight measurement method using a strain gage, detect the amount of remaining solution and perform an alarm function.

However, in the above-described conventional designs for the injection of Ringer's solution can calculate the amount of remaining solution and perform a function of giving an alarm, but the output range of the alarm is limited to the inside of a hospital room. Therefore, it is impossible to completely remove the inconvenience of forcing a nurse or caregiver, observing the injection of the Ringers solution, to always stay in a hospital room or to periodically check the status of a patient and the Ringers solution when an injection of Ringer's solution is given to the patent suffering inconvenience in action.

### SUMMARY OF THE INVENTION

Accordingly, the present invention has been made keeping in mind the above problems occurring in the prior art, and an object of the present invention is to provide a system for monitoring the injection of Ringer's solution, which measures the weight of Ringer's solution being injected and/or the injection status of the Ringer's solution, transmits the measurement results to a computer, an alarm device or a mobile terminal placed at a remote place, and displays the measurement results, so that the injection status and replacement status of the Ringer's solution can be monitored at a remote place, thus removing the inconvenience of forcing a nurse or caregiver to frequently observe the injection status of the Ringer's solution at the time of giving an injection of the Ringer's solution to a patient.

In order to accomplish the above object, the present invention provides a system for monitoring an injection of Ringer's solution, comprising a status detection device for measuring a weight of Ringer's solution, converting weight information of the Ringer's solution into a data signal, and transmitting the data signal; a first line interface device for receiving the data signal transmitted from the status detection device, performing data conversion with respect to the data signal to allow identifier information required to identify the status detection device to be included in the data signal, and outputting the converted data signal to a communication line; a second line interface device for receiving the data signal input from the first line interface device through the communication line, and outputting the data signal to a remote monitoring device connected thereto; and the remote monitoring device for decoding the data signal output from the second line interface device and outputting weight data of the Ringer's solution according to the identifier information.

The communication line may be a power line network, a leased line network using a wired cable, or a telephone line network connected to a Public Switched Telephone Network (PSTN) or an intercom network, and the first and second line interface devices can be selectively constructed using power line modems, leased line modems or telephone line modems according to the type of communication line.

The weight of Ringer's solution hung to give an injection of the Ringer's solution to a patient is measured by a status detection device 100 and converted into an infrared signal, and the infrared signal is output. Then, a first line interface device 200 receives the infrared signal output from the status detection device 100, encodes or modulates the infrared signal to add identifier information, including the name of a patent being given an injection of the Ringer's solution, the location of the patient or the type of Ringer's solution, amplifies the encoded or modulated signal to prevent data loss during data transmission, and outputs the amplified signal to a communication line 500. The information on the status of Ringer's solution output as described above is decoded or demodulated by a second line interface device 300, and transmitted to a remote monitoring device 400, so that the Ringer's solution status information is output from the remote monitoring device 400 according to an identifier. Accordingly, the inconvenience of forcing a nurse to periodically visit a patients hospital room and observe the injection status of Ringer's solution during injection of the Ringer's solution can be removed.

### BRIEF DESCRIPTION OF THE DRAWINGS

FIG. 1 is a view showing the construction of a system according to the present invention;

FIG. 2 is a view showing the construction of the system of FIG. 1 according to an embodiment of the present invention;

FIG. 3 is a block diagram of a status detection device of FIG. 2;

FIG. 4 is a perspective view showing the construction of a switch of FIG. 2

FIG. 5 is a block diagram of first and second line interface devices;

FIG. 6 is a block diagram of a line interface unit applied to a power line;

FIG. 7 is a block diagram of a line interface unit applied to a leased line;

FIG. 8 is a block diagram of a line interface unit applied to a PSTN;

FIG. 9 is a block diagram of a line interface unit applied to an intercom line; and

FIG. 10 is a view showing the construction of the status detection device using a solution monitoring device.

### DETAILED DESCRIPTION OF THE INVENTION

Hereinafter, embodiments of the present invention will be described in detail with reference to the attached drawings.

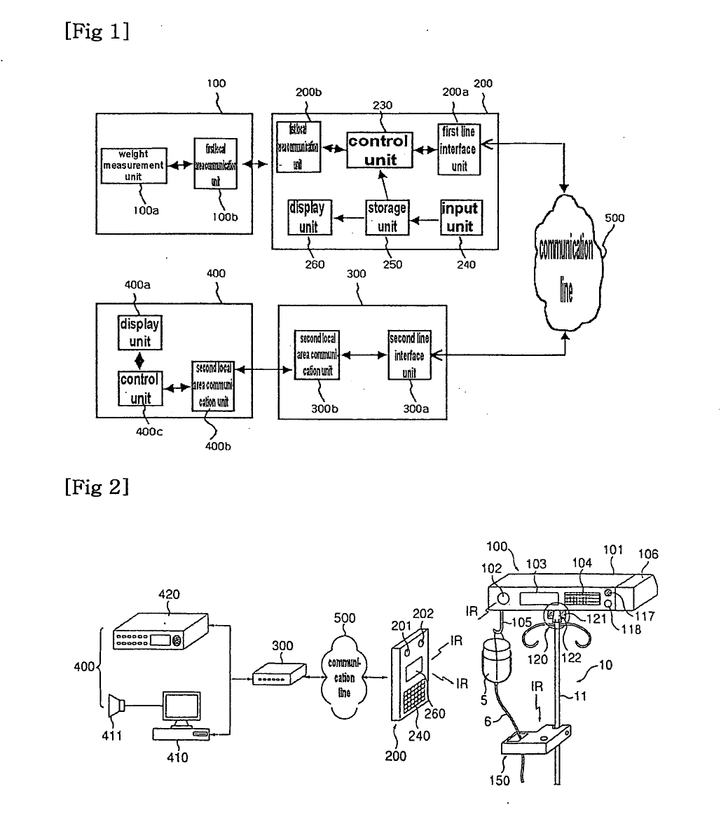

FIG. 1 is a block diagram showing the construction of a system according to the present invention. With reference to FIG. 1, the components and construction of the system according to the present invention are described in detail.

As shown in FIG. 1, a status detection device includes a weight measurement unit 100a or status measurement unit for measuring the weight of Ringer's solution and/or the injection status of the Ringer's solution, converting the measurement results into a data signal and outputting the data signal; and a first local area communication unit 100b for performing data communication with a first line interface device 200 to transmit the data signal, output from the weight measurement unit 100a and/or the status measurement unit, to the first line interface device 200.

The first line interface device 200 includes a first local area communication unit 200b for performing data communication with the status detection device; and a first line interface unit 200a connected to a communication line 500 to perform data communication with a second line interface device 300 located at a remote place through the communication line. The first line interface device 200 may further include an input unit 240 for receiving identifier information to identify the status detection device 100; a storage unit 250 for storing therein the identifier information received from the input unit; a display unit 260 for displaying the identifier information; and a control unit 230 for performing data conversion for the data signal, input from the first local area communication unit, to allow the identifier information to be included in the data signal, and outputting the converted data signal to the first line interface unit. However, the input unit 240, the storage unit 250, the display unit 260 and the control unit 230 may be constructed in the status detection device 100.

The identifier information may be composed of pieces of information about the floor where the status detection device is placed, the hospital room number, and the bed number, and may further include information about the type and weight of Ringer's solution, etc.

The second line interface device 300 includes a second local area communication unit 300a for performing local area data communication with the remote monitoring device 400; and a second line interface unit 300b connected to the communication line to perform data communication with the first line interface device 200 through the communication line.

The remote monitoring device 400 includes a second local area communication unit 400b for performing local area data communication with the second line interface device 300; a control unit 400c for extracting the identifier of the status detection device and weight information from the data received through the second local area communication unit 400b and outputting the extracted information; and a display unit 400a for classing and displaying the weight information of Ringer's solution output from the control unit 400c according to an identifier. The remote monitoring device 400 may be software installed in a Personal Computer (PC) or an independent display device.

In the entire system of the present invention implemented using the above-described respective components, the status detection device 100 and the first line interface device 200 are installed in a hospital room so as to perform local area data communication through the first local area communication units 100b and 200b, and the remote monitoring device 400 and the second line interface device 300 are installed in a nurse room at a remote place so as to perform local area data communication through the second local area communication units 300b and 400b. Further, the present invention can be constructed so that the first line interface unit 200a of the first line interface device 200 is connected to the communication line 500 through an interface terminal of a communication line in the hospital room, and the second line interface unit 300a of the second line interface device 300 is connected to the communication line 500 through an interface terminal of a communication line placed at the remote place, such as the nurse room, thus intermediating data communication between the status detection device 100 and the remote monitoring device 400.

A plurality of status detection devices 100 and first line interface devices 200 may be installed in each hospital room where patients to be injected with Ringer's solution stay. The remote monitoring device 400 can be installed for each management area so that objects to be managed may be classified according to a floor number, hospital room number, or patient name to be managed, and only information on objects being managed may be displayed. In this case, the remote monitoring device 400 extracts identifier information, allocated by the status detection device 100 or the first line interface device 200, from the data having the weight information or injection status information of Ringer's solution that is measured by the Ringer's solution weight measurement unit 100a and transmitted from the status detection device 100 through the communication line, classifies the weight information or injection status information that is the management object information set to be managed by the remote monitoring device 400 depending on floor number, hospital room number or bed number according to the identifier, and displays the classified information.

The installation and driving schemes of the remote monitoring device 400 of the present invention are not limited to the above description but can be variously modified in such a way that a main remote monitoring device is further installed in the center of a hospital to monitor the injection status of Ringer's solution of the entire hospital, or the remote monitoring device is implemented using a portable terminal to allow nurses to conveniently carry the remote monitoring device.

For data communication between respective components implemented in the above-described system of the present invention, various wired/wireless data communication methods can be used. However, it is preferable to remove interference with medical instruments according to the characteristics of hospitals. In particular, the data communication between the first line interface device 200 and the status detection device 100 is preferably implemented using infrared communication or direct wired communication in consideration of the congestion of hospital rooms, but the data communication is not limited to the infrared or wired communication.

FIGS. 2 to 5 are views showing the construction of a system constructed to allow the status detection device 100 and the first line interface device 200 to perform local area infrared communication according to an embodiment of the present invention. Hereinafter, with reference to FIGS. 2 to 5, the construction of the system, constructed to allow the status detection device 100 and the first line interface device 200 to perform local area infrared communication according to the embodiment of the present invention, is described in detail.

As shown in FIG. 2, in the system of the present invention, there are installed, in a hospital room where a patent stays, the status detection device 100 adapted to measure the weight of Ringer's solution 5 and output the measured weight as an infrared signal, and the first line interface device 200 connected to the communication line, such as a power line, a leased line or telephone line, to receive the infrared signal output from the status detection device 100 and transmit the infrared signal to a remote place. Furthermore, the other end of the communication line is connected to the second line interface device 300.

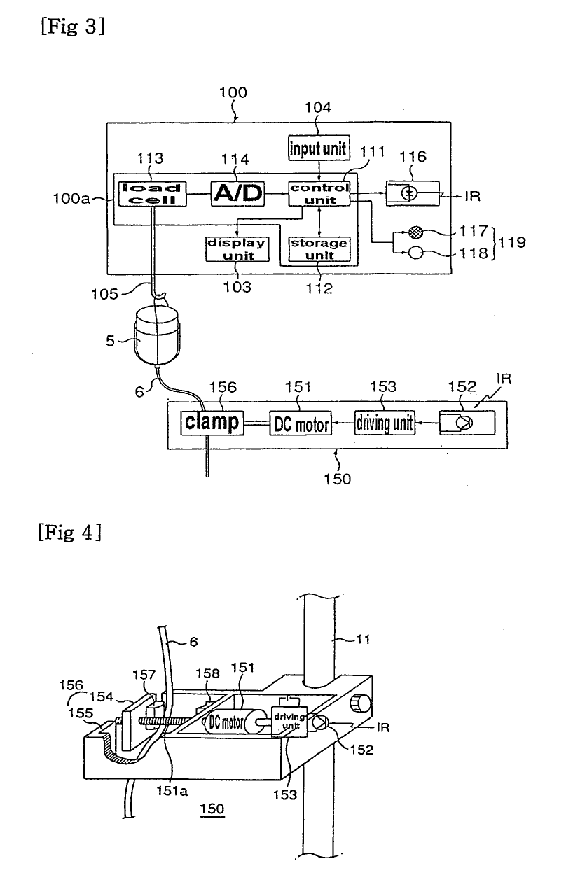

FIG. 3 is a block diagram showing the construction of the status detection device 100 according to an embodiment of the present invention.

The status detection device 100 of FIG. 3 is related to a Ringer's solution hanger having a weight measurement unit. The status detection device 100 includes a load cell 113, to which a Ringer's solution holder 105 allowing the Ringer's solution 5 to be hung thereon is attached, and which outputs an electrical signal including the weight information of the Ringer's solution; a control unit 111 for arithmetically processing the electrical signal output from the load cell 113 and calculating the weight of the Ringer's solution; and an infrared transmission unit 116 for converting the electrical signal having the weight information of the Ringer's solution output from the control unit 111 into an infrared signal and transmitting the infrared signal. Further, an Analog/Digital (AND) converter 114 for converting the analog electrical signal output from the load cell into a digital signal to execute digital data processing may be disposed between the load cell 113 and the control unit 111.

In this case, the load cell 113, the A/D converter 114 and the control unit 111 constitute the Ringer's solution weight measurement unit 100a of FIG. 1, and the infrared transmission unit 116 is an embodiment of the first local area communication unit 100b of FIG. 1. The constructions of the weight measurement unit 100a and the first local area communication unit 100b of the present invention are not limited to the embodiments, as described above.

The load cell 113 is configured to convert the variations in weight into an electrical signal and output the electrical signal, and includes an elastic body elastically transformed depending on a weight, a strain gage attached to an outer circumference of the elastic body, and a resistance detection circuit, such as a Wheatstone bridge, for outputting an analog electrical signal, such as a voltage or current corresponding to the transformation of the elastic body, through the strain gage. The construction of the load cell 113 is well known, so the details of its construction are omitted.

The load cell 113 having the above construction is operated so that, if the Ringer's solution 5 is hung on the Ringer's solution holder 105, the elastic body is transformed due to the weight of the Ringer's solution 5, the internal resistance of the strain gage varies with the transformation of the elastic body, and then a ratio of the output voltage to input voltage of the resistance detection circuit is output as a value reflecting the variations in internal resistance.

The control unit 111 compares the input and output voltages of the resistance detection circuit to each other, calculates variations in internal resistance of the strain gage, and calculates variations in force applied to the elastic body on the basis of the resistance variations, thus detecting the weight of the Ringer's solution or the amount of remaining solution. Further, the control unit 111 transmits the Ringer's solution weight or the amount of remaining Ringer's solution to the infrared transmission unit 116, or to the first line interface device through a wired line.

The status detection device 100 of FIG. 2 includes a rectangular parallelepiped-shaped box-type casing 101 having an arch-shaped upper surface, and the Ringer's solution holder 105 connected to the load cell 113 and formed on a portion of the bottom surface of the casing 101. The status detection device 100 is illustrated as a folder detachable device that is attached to or detached from the folder 10 by a tube 122 projected downward and a compression nut 121, but the status detection device 100 is not limited to this structure, and it can be constructed to be a folder-integration type in which the folder 10 is formed on the bottom of the casing 101 without requiring the compression nut 121 and the tube 122.

Further, if the status detection device 100 is constructed to be allocated an identifier and identified thereby, and to have a function of giving an alarm when the injection of Ringer's solution is completed, the status detection device 100 may further include a display unit 103, such as a Liquid Crystal Display (LCD), an input unit 104, such as a keypad, to input identifier information required to identify the status detection device 100, information of the amount of initial solution injected, and threshold amount information, and an alarm output unit 119 comprised of a speaker 117 and a Light emitting Diode (LED) 118 to indicate the termination of the injection of the Ringer's solution.

In this case, the control unit 111 may be constructed to display the measured Ringer's solution weight or amount of remaining solution on the display unit 103, or output an alarm when an amount of solution equal to or greater than a certain level is injected. Further, all or part of the data, such as the identifier information allocated to the status detection device, the information on the amount of initial solution, and the threshold amount information, may be constructed to be attached or detached in the form of a memory card, etc.

If the remote monitoring device 400 is provided with a function of generating a signal blocking the injection of the Ringer's solution, the status detection device 100 may further include a switch 150, which is a selective component for blocking the injection of the Ringer's solution in response to a solution blocking signal transmitted from the remote monitoring device 400. Reference numeral 150 of FIG. 1 shows an example of the exterior shape of the switch 150, and FIGS. 3 and 4 show the detailed construction thereof.

As shown in FIGS. 3 and 4, the switch 150 can be fixed on the axis 11 of the folder 10 and is comprised of a Direct Current (DC) motor 151, an infrared sensor 152, a driving unit 153 for driving the DC motor 151 in response to a signal input from the infrared sensor 152, and a clamp 156 attached to the shaft 151 a of the DC motor 151 with a solution tube 6 inserted thereto to block the injection into the solution tube 6.

The damp 156 includes an external casing 155 having a reception projection 158 attached to the outside of the DC motor 151 and provided with a depression formed therein; and a movable member 154 located in the external casing 155 while remaining engaged with the shaft of the DC motor 151 so as to be horizontally movable along the shaft depending on the rotational direction of the shaft of the DC motor 151, and provided with a compression projection 157 having a protrusion to be brought into tight contact with the reception projection 158. The solution tube 6 is fixed between the compression projection 157 and the reception projection 158.

The driving unit 153 drives the DC motor 151 when an alarm signal is output from the status detection device 100 or when a solution injection blocking signal transmitted from the remote monitoring device 400 is input from the first line interface device 200. Therefore, with the driving of the DC motor 15, the solution tube 6 is tightly fastened to the damp, thus blocking the injection of the solution.

The switch 150 and the damp 156 of FIGS. 3 and 4 are embodiments of the present invention, and can be variously modified and implemented for the convenience of users.

In the meantime, the status detection device of FIG. 10 shows an example implemented with a solution monitoring device. In order to measure an injection rate or injection degree of the Ringer's solution 5 dropping from a chamber 160 of Ringer's solution after the solution monitoring device is fitted over the chamber 160, the solution monitoring device includes a ring-shaped chamber fitting unit 161 fitted over the chamber, an infrared LED 162 installed on the inner circumference of the chamber fitting unit to emit an infrared ray, a phototransistor 163 installed on the inner circumference of the chamber fitting unit to be opposite to the infrared LED so as to detect the infrared ray emitted from the infrared LED, and a power line 164 for supplying power to both the infrared LED and the phototransistor and transmitting a signal generated from the phototransistor.

This process is described in detail as follows. If the infrared LED emits light and projects an infrared ray, the phototransistor measures the amount of light projected and received from the infrared LED. If the solution drops, the reception of the infrared ray is interrupted due to the solution. The phototransistor examines whether the reception of the infrared ray is interrupted, and transmits the examination result value to the infrared transmission unit or the first line interface device through the power line.

Such a solution monitoring device can be used together with the weight measurement unit, or used independently, and performs the same function as the status detection device in that it monitors the injection of Ringer's solution given to a patent. With reference to FIG. 2 again, the embodiment of the present invention is described. The above-described remote monitoring device 400 is implemented with a PC 410 or independent alarm indication device 420 installed with software allowing the Ringer's solution weight, the amount of remaining solution and alarm data, which are transmitted from the status detection device 100, to be displayed.

Further, the above-described remote monitoring device 400 can be constructed to output a switch off signal for stopping the injection of solution. In this case, when the switch off signal is output from the first line interface device 200 or the infrared transmission/reception unit 116 of the first line interface device, the driving unit 153 rotates the DC motor 151 to change the position of the movable member 154, thus blocking the injection of the solution.

The remote monitoring device 400 may further include an alarm output unit 411 comprised of a speaker and/or a LED that output an alarm signal when the injection of the Ringer's solution has been completed.

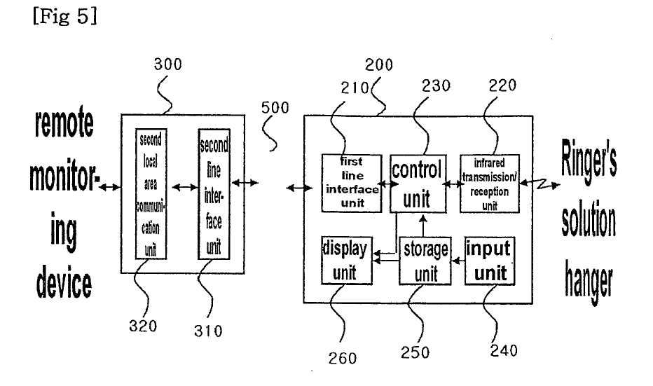

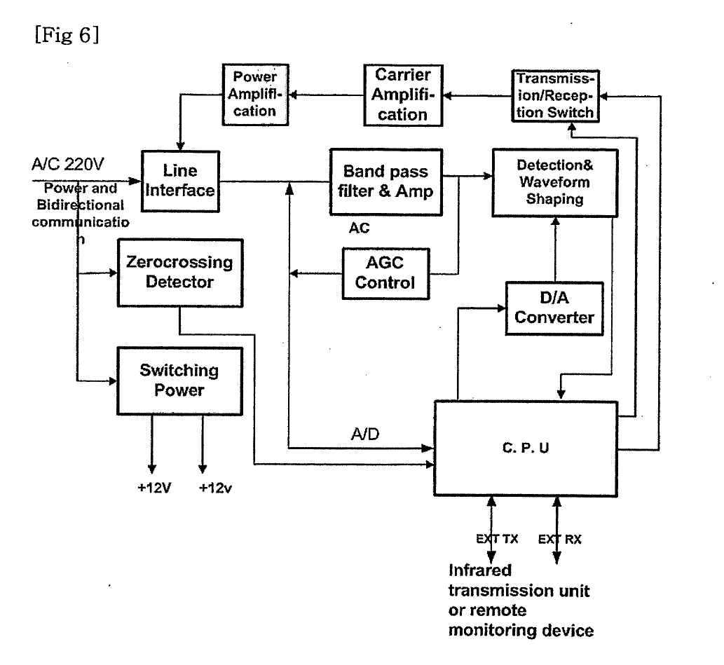

FIG. 5 is a block diagram showing the internal construction of the first and second line interface devices 200 and 300 of FIG. 2.

The first line interface device 200 may be comprised of a first line interface unit 210 for controlling the transmission of data through the communication line, and a control unit 230 for decoding a signal transmitted from the status detection device 100 to the first line interface device and outputting the decoded signal.

Further, as shown in FIG. 5, the first line interface device 200 may include the first line interface unit 210 for controlling the transmission of data through the communication line; an infrared transmission/reception unit 220 for performing infrared data communication with the status detection device; and the control unit 230 disposed between the infrared transmission/reception unit 220 and the first line interface unit 210 to convert a data signal, received from the status detection device 100 through the infrared transmission/reception unit 220, into a predetermined data format, output the converted data to the first line interface unit 210, decode a data transmission packet signal received from the first line interface unit 210, and output the decoded signal to the infrared transmission/reception unit 220.

Further, the first line interface device 200 may selectively include an input unit 240 for receiving identifier information used to identify the status detection device 100, such as floor number, hospital room number, and bed number; a storage unit 250 for storing therein the data received from the input unit; and a display unit 260 for displaying the Ringer's solution weight data, transmitted from the status detection device 100, and the identifier information. In this case, it is preferable that the control unit 230 is constructed to include the identifier information in data to be transmitted, convert a data format, and output the converted data to the first line interface unit 210.

In this case, the input unit 240, the storage unit 250, the display unit 260 and the control unit 230 that are constructed to input the identifier information used to identify the status detection device 100 can be selectively constructed in the status detection device 100. In this case, the control unit 111 of the status detection device 100 is constructed to encode the Ringer's solution weight data, converted into a digital signal, to have the identifier, and output the encoded results to the infrared transmission unit 116, as described above.

The second line interface device 300 includes a second line interface unit 310; and a second local area communication unit 320 for performing data communication with 15 the remote monitoring device 400.

The above-described line interface units 210 and 310 have the same construction, and use the same name.

As described above, in the present invention, the communication line may be a power line network, a leased line network using a wired cable, or a telephone line network connected to a PSTN or an intercom network.

Therefore, the first and second line interface devices can be selectively implemented with power line modems, leased line modems, or telephone line modems according to the communication line so that they are connected to opposite ends of the communication line to perform modulation/demodulation, encoding/decoding and/or amplification with respect to data signals transmitted and received through the communication line and to transmit the data to a remote place without causing data loss during a transmission process through the communication line.

That is, the first and second line interface devices 200 and 300 may include the first and second line interface units having various constructions shown in FIGS. 6 to 9 according to the type of applied communication line.

FIG. 6 illustrates an example of the construction of the first and second line interface units implemented with power line modem modules to allow the first and second line interface devices 200 and 300 to be operated as power line modems connected to a power line. The first and second line interface units perform D/A conversion, modulation into a carrier, and amplification with respect to a digital signal having data that are to be transmitted through the power line as the communication line, and then output the performance results to the communication line. Further, the first and second line interface units perform frequency filtering, detection and waveform shaping, and AND conversion with respect to a modulated signal received from the power line, and then output the performance results to terminal devices connected thereto. In the present invention, in the case of the first line interface device, a corresponding signal is converted into an infrared signal and then output to the status detection device, while, in the case of the second line interface device, a corresponding signal is output to a cable or communication scheme connected to the remote monitoring device in a wired or wireless manner.

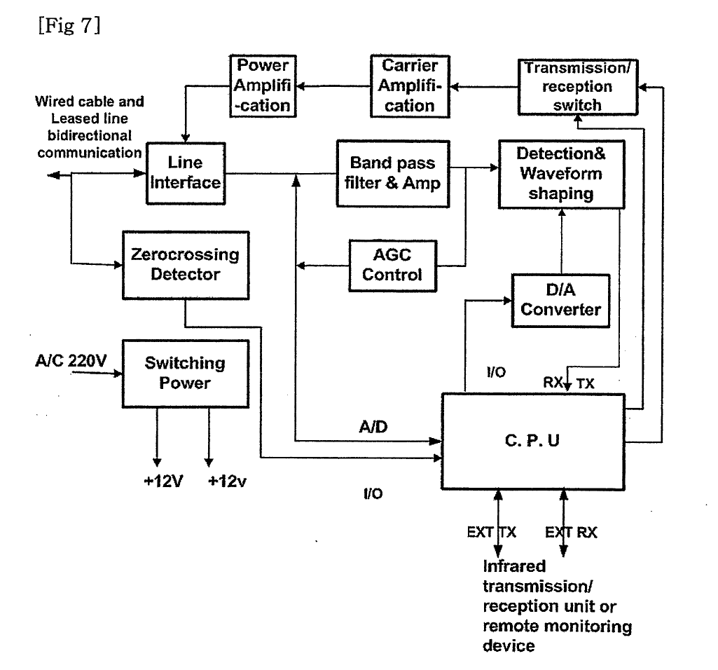

FIG. 7 illustrates an example of the construction of the first and second line interface units implemented with leased line modem modules to allow the first and second line interface units 200 and 300 to be connected to a wired cable or leased line. The first and second line interface units execute signal processing identical to that of FIG. 6, and are different from that of FIG. 6 in that the communication line is a wired cable, such as a coaxial cable or Unshielded Twisted Pair (UTP) cable, not a power line.

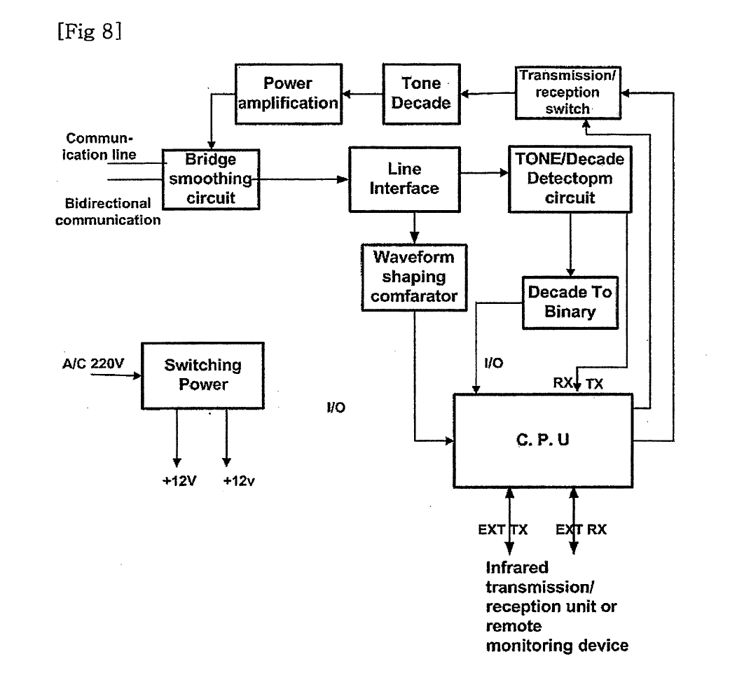

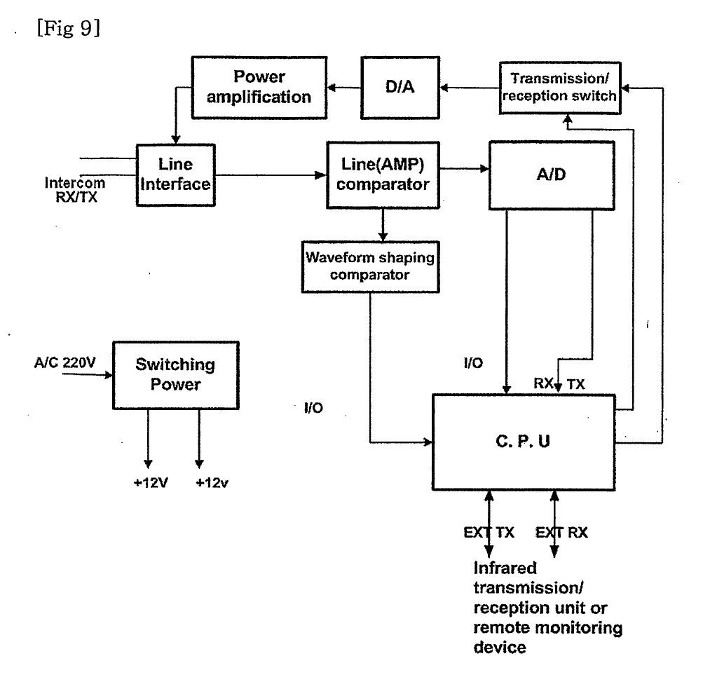

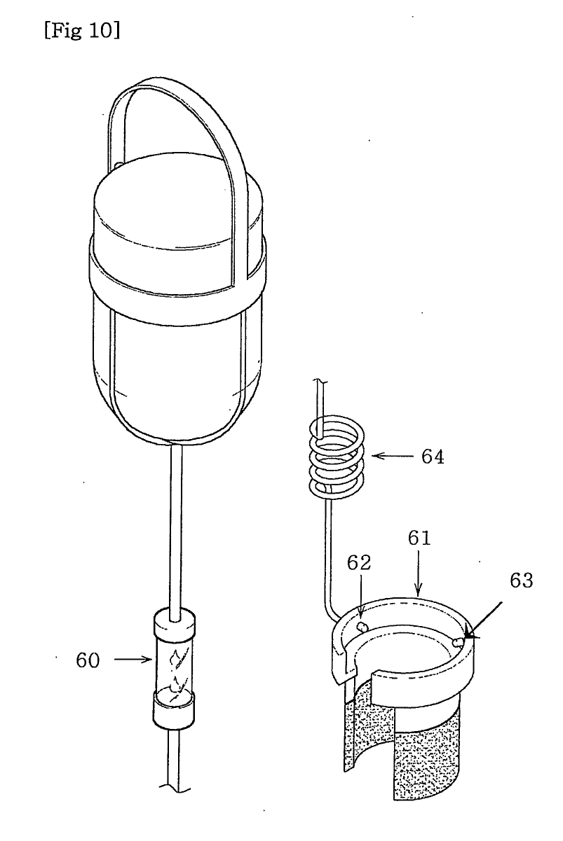

FIG. 8 illustrates an example of the construction of the first and second line interface units implemented with telephone line modem modules for PSTN to allow the first and second line interface devices 200 and 300 to be connected to the PSTN. FIG. 9 illustrates an example of the construction of the first and second line interface units implemented with intercom line modem modules to allow the first and second line interface devices 200 and 300 to be connected to the intercom line.

The first and second line interface units of FIGS. 6 to 9 are generally constructed in power line modems, telephone line modems for PSTN or intercom lines, and leased line modems, such as cable modems, so that a detailed description thereof is omitted.

The present invention transmits information on a time to replace Ringer's solution to a remote place, such as a nurse room, and displays the replace time, thus removing the inconvenience of forcing a nurse or caregiver to periodically observe the injection process of the solution during the injection of the solution at the time of giving an injection of Ringer's solution using a Ringer bottle or pack.

Further, the present invention uses infrared communication and power line modems, thus facilitating the installation and removal of the system, and solving a problem of causing malfunction or failure of medical instruments when Radio Frequency (RF) signals are used.

1. A system for monitoring an injection of Ringer's solution, comprising:
a status detection device for measuring a weight of Ringer's solution, converting weight information of the Ringer's solution into a data signal, and transmitting the data signal;
a first line interface device for receiving the data signal transmitted from the status detection device, performing data conversion with respect to the data signal to allow identifier information required to identify the status detection device to be included in the data signal, and outputting the converted data signal to a communication line;
a second line interface device for receiving the data signal input from the first line interface device through the communication line, and outputting the data signal to a remote monitoring device connected thereto; and
the remote monitoring device for decoding the data signal output from the second line interface device and outputting weight data of the Ringer's solution according to the identifier information.

2. The Ringer's solution injection monitoring system according to claim 1, wherein the status detection device comprises:
a load cell to which a Ringer's solution holder allowing the Ringer's solution to be hung thereon is attached, the load cell outputting an electrical signal having the weight information of the Ringer's solution;
a control unit for arithmetically processing the electrical signal output from the load cell and calculating the weight of the Ringer's solution; and
a first local area communication unit for transmitting the weight information of the Ringer's solution output from the control unit to the first line interface device.

3. A system for monitoring an injection of Ringer's solution, comprising:
a status detection device fitted over a chamber of the Ringer's solution to determine whether the Ringer's solution dropping from the chamber is injected and/or to detect an injection rate thereof, converting detected values into a data signal, and transmitting the data signal;
a first line interface device for receiving the data signal transmitted from the status detection device, performing data conversion with respect to the data signal to allow identifier information required to identify the status detection device to be included in the data signal, and outputting the converted data signal to a communication line;
a second line interface device for receiving the data signal input from the first line interface device through the communication line, and outputting the data signal to a remote monitoring device connected thereto; and
the remote monitoring device for decoding the data signal output from the second line interface device and outputting weight data of the Ringer's solution according to the identifier information.

4. The Ringer's solution injection monitoring system according to claim 3, wherein the status detection device comprises:
a ring-shaped chamber fitting unit fitted over the chamber of the Ringer's solution so as to measure an injection rate or injection degree of the Ringer's solution dropping from the chamber,
an infrared Light Emitting Diode (LED) installed on an inner circumference of the chamber fitting unit to emit an infrared ray;
a phototransistor installed on the inner circumference of the chamber fitting unit to be opposite to the infrared LED so as to detect the infrared ray emitted from the infrared LED; and
a power line for supplying power to both the infrared LED and the phototransistor and transmitting a signal generated from the phototransistor.

5. The Ringer's solution injection monitoring system according to claim 1 or 3, wherein the first line interface device comprises:
a first local area communication unit for performing data communication with the status detection device;
a first line interface unit connected to the communication line so as to perform data communication with the second line interface device located at a remote place through the communication line;
an input unit for receiving the identifier information required to identify the status detection device;
a storage unit for storing the identifier information input from the input unit;
a display unit for displaying the identifier information; and
a control unit for performing data conversion with respect to the data signal input from the first local area communication unit to allow the identifier information to be included in the data signal, and for outputting the converted data signal to the first line interface unit.

6. The Ringer's solution injection monitoring system according to claim 1 or 3, wherein the second line interface device comprises:
a second line interface unit connected to the communication line to perform data communication with the first line interface device through the communication line; and
a second local area communication unit for performing local area data communication with the remote monitoring device.

7. The Ringer's solution injection monitoring system according to claim 1 or 3, wherein the remote monitoring device comprises:
a second local area communication unit for performing local area data communication with the second line interface device;
a control unit for extracting identifier information of the status detection device and weight information of Ringer's solution from data received from the second local area communication unit, and outputting the identifier information and weight information; and
a display unit for displaying the weight information of the Ringer's solution output from the control unit according to an identifier.

8. The Ringer's solution injection monitoring system according to claim 1 or 3, wherein the status detection device further comprises a switch for blocking the injection of the Ringer's solution.

9. The Ringer's solution injection monitoring system according to claim 2, wherein the status detection device comprises:
an input unit for receiving identifier information to identify the status detection device;
a storage unit for storing the identifier information input from the input unit; and
a display unit for displaying the identifier information, wherein the control unit performs data conversion with respect to the Ringer's solution weight information signal input from the load cell so as to allow the identifier information to be included in the Ringer's solution weight information signal, and outputs the converted signal to the first local area communication unit.

10. The Ringer's solution injection monitoring system according to claim 2 or 5, wherein the first line interface unit is implemented with any one of a power line modem module, a leased line modem module, a telephone line modem module for a Public Switched Telephone Network (PSTN), and a telephone line modem module for an intercom line.

11. The Ringer's solution injection monitoring system according to claim 2 or 5, wherein the first local area communication unit is implemented with any one of a wired data communication interface for wired communication, and a wireless data communication interface for wireless communication.

12. The Ringer's solution injection monitoring system according to claim 5, wherein the identifier is comprised of one or more of floor number of a hospital, hospital room number, bed number and patient's name.

13. The Ringer's solution injection monitoring system according to claim 5, wherein the storage unit for storing the identifier information is a memory card.

14. The Ringer's solution injection monitoring system according to claim 6 or 7, wherein the second local area communication unit is implemented with any one of a wired data communication interface for wired communication, and a wireless data communication interface for wireless communication.

15. The Ringer's solution injection monitoring system according to claim 6 or 7, wherein the second line interface unit is implemented with any one of a power line modem module, a leased line modem module, a telephone line modem module for a PSTN, and a telephone line modem module for an intercom line.

16. The Ringer's solution injection monitoring system according to claim 7, wherein the remote monitoring device has management areas, classified according to a floor number, hospital room number, the patents name or bed number and designated thereby, and displays only the weight data of corresponding Ringer's solution belonging to a designated management area using the identifier information of the data input through the communication line.

17. The Ringer's solution injection monitoring system according to claim 11, wherein the first local area communication unit is an infrared transmission/reception unit.
